(19) United States
(12) Patent Application Publication
(10) Pub. No.: US 2023/0212427 A1
(43) Pub. Date: Jul. 6, 2023

(54) METHOD FOR TREATING A SURFACE AND USE OF COLLAGEN HYDROLYSATE FOR TREATING A SURFACE

(71) Applicant: GELITA AG, Eberbach (DE)

(72) Inventors: Berthold KÖHLER, Neckargemünd (DE); Matthias REIHMANN, Hemsbach (DE); Peter REITER, Meckesheim (DE); Nina RITTEREISER, Waldbrunn (DE); Ceren YÜCE, Walldorf (DE)

(21) Appl. No.: 18/119,966

(22) Filed: Mar. 10, 2023

Related U.S. Application Data
(63) Continuation of application No. PCT/EP2021/068937, filed on Jul. 8, 2021.

(30) Foreign Application Priority Data
Sep. 11, 2020 (DE) 10 2020 123 736.7

(51) Int. Cl. C09D 189/06; C09D 5/14; A61L 2/18

(52) U.S. Cl. CPC C09D 189/06; C09D 5/14; A61L 2/18

(57) ABSTRACT

The present invention relates to a method for treating a surface in order to improve the removability of microorganisms from the surface, comprising the steps: —applying a treatment solution onto the surface, wherein the treatment solution comprises an aqueous solution of a collagen hydrolysate; and—allowing the surface to dry. The present invention further relates to the use of collagen hydrolysate for treating a surface in order to improve the removability of microorganisms from the surface.

### FIELD OF THE INVENTION

The present invention relates to a method for treating a surface in order to improve the removability of microorganisms from the surface.

### BACKGROUND OF THE INVENTION

The contamination of surfaces with microorganisms, in particular bacteria and viruses, represents a problem in various settings in daily life and in industry since diseases can be transmitted by a large number of pathogenic microorganisms via contact infection, or since foods can be contaminated by coming into contact with such a surface. Typically, microorganisms on surfaces cannot or can only incompletely be removed, killed or deactivated by cleaning with water or surfactants.

Therefore, biocides are often used as disinfectant agents to reduce the burden of microorganisms from surfaces. A variety of chemical compounds with a biocidal effect (e.g. a bacteriocidal effect) are known, among them, for example, alcohols, isothiazolinones, quarternary ammonium compounds and formaldehyde-releasing compounds. However, the use of such disinfectant agents, in particular when used frequently, can be problematic or undesirable with regard to health risks (e.g. from contact allergies) or with respect to environmental load.

In addition, the aforementioned disinfectant agents have only a one-time effect whenever they are used. In addition, surface treatment agents having a long-term effect against microorganisms are also known, for example, colloidal or nanoparticulate silver, specific copper compounds or nanoparticulate titanium dioxide. However, these substances are also more problematic with regard to health risks and, in the case of titanium dioxide, due to its mode of action (formation of radicals by UV light), even plastics surfaces can be attacked.

### BRIEF SUMMARY OF THE INVENTION

It is an object of the invention to propose a method through the use of which the contamination of surfaces by microorganisms can easily be reduced in the long term.

This object is achieved according to the invention with the method of the type mentioned in the introduction in that the method comprises the following steps:
applying a treatment solution onto the surface, wherein the treatment solution comprises an aqueous solution of a collagen hydrolysate; and
allowing the surface to dry.

### BRIEF DESCRIPTION OF THE SEVERAL VIEWS OF THE DRAWING(S)

FIGS. 1 to 8 show: microscopy images of E. coli bacteria on slides.

### DETAILED DESCRIPTION OF THE INVENTION

It has been unexpectedly discovered that a surface treated according to the invention which, after the treatment, is contaminated with microorganisms, can be more easily cleared of said microorganisms than an untreated surface. In particular, the method according to the invention has the result that a larger proportion of the microorganisms can be removed again by rinsing with water than in the case of an untreated surface.

The collagen hydrolysate applied forms a hydrophilic layer on the treated surface after drying, which is typically a few nanometres thick. Since collagen hydrolysate, as a protein, represents a potential nutrient substrate for microorganisms, it is entirely surprising that the method according to the invention contributes to microorganisms being able to be removed from surfaces more easily. Without being bound to a theory, the inventors assume that an adhesion of microorganisms to the collagen hydrolysate layer which swells up in the presence of water is made more difficult so that cleaning them off with water (or a cleaning agent) is facilitated. Furthermore, during subsequent cleaning, parts of the layer become detached together with the microorganisms.

The method according to the invention can thus make a contribution to at least reducing the use of chemical disinfectant agents (biocides) in the cleaning of surfaces.

Collagen hydrolysate which is used in the context of the method according to the invention is a decomposition product of the animal connective tissue protein collagen. In contrast to gelatin which is also obtained from collagen, due to its low molecular weight collagen hydrolysate is water-soluble even at room temperature and does not form a gel.

Preferably, the collagen hydrolysate is produced by enzymatic hydrolysis of a collagen-containing starting material. For the hydrolysis, in particular microbial or plant-sourced endopeptidases and exopeptidases can be used. The molecular weight of the collagen hydrolysate produced can be influenced by means of the selection of the peptidases and the hydrolysis conditions.

The collagen-containing starting material is typically selected from the skin or bone of vertebrates, in particular cattle or pigs. However, the use of starting materials from other mammals, birds or fish is also conceivable.

As an alternative to enzymatic hydrolysis, the collagen hydrolysate can be produced in the context of the invention by means of recombinant gene expression. By means of the use of natural collagen sequences, in particular from cattle or pigs and their expression in genetically modified cells (e.g. yeasts, bacteria or plant cells, in particular tobacco), products which are substantially identical to the hydrolysis products of the corresponding collagen-containing raw materials can be produced. It is possible therein to obtain a closer and/or exactly prescribed molecular weight distribution.

The collagen hydrolysate preferably has a mean molecular weight from approximately 1,000 to approximately 7,000 Da, more preferably from approximately 2,000 to approximately 5,000 Da. These details relate to the mean molecular weight which is determined by means of gel permeation chromatography. As already mentioned, collagen hydrolysate in this molecular weight range has good solubility in cold water.

The treatment solution preferably comprises approximately 0.5 to approximately 8 wt % of the collagen hydrolysate, more preferably approximately 2 to approximately 6 wt %.

According to an advantageous embodiment of the method, the treatment solution is applied onto the surface in a quantity from approximately 10 to approximately 50 ml/m², more preferably in a quantity from approximately 20 to approximately 30 ml/m².

The treatment solution can be spread onto the surface, e.g. with a sponge or a cloth, or sprayed onto the surface.

The microorganisms, the removability of which can be improved by the method according to the invention, comprise, in particular, bacteria, fungi, algae and viruses. Although the last-mentioned are not, in the majority view, living beings, they are often included—as they are in the context of the present invention—under the general heading of microorganisms. All the types of microorganisms mentioned contain pathogens or can lead to the decaying of foods that come into contact with a contaminated surface.

In a preferred embodiment of the invention, the microorganisms are bacteria, in particular faecal bacteria such as, for example, Escherichia coli.

According to a preferred embodiment of the invention, the treatment solution contains no biocides. Biocides are not necessary for the fundamental effect of the method, since the removability of the microorganisms with water or a cleaning agent is already improved. In particular, according to the invention, a treatment solution can be used which, apart from the collagen hydrolysate, comprises no further active ingredients, in particular no surfactants. A treatment solution of this type is entirely harmless from a health and ecological standpoint and can therefore be often used. This can then be combined with the use of a disinfecting agent at greater intervals of time.

According to a further advantageous embodiment of the invention, the treatment solution comprises further active agents which are preferably selected from biocides, in particular bactericides, surfactants, phosphates, preserving agents, perfumes and/or excipients. In this variant, the collagen hydrolysate is, in principle, added to a common cleaning and/or disinfectant agent. By means of the application of this treatment solution, microorganisms situated on the surface are removed, killed and/or deactivated and, at the same time, the inventive effect occurs that the efficiency of subsequent cleaning processes of the surface is improved with regard to the removal of microorganisms.

The method according to the invention can be applied, in principle, to all surfaces, although the surfaces are preferably smooth. The method is suitable, in particular, for the treatment of surfaces made of glass, plastics or metal.

The method according to the invention can be used in a wide range of areas, for example, in the domestic sphere, in industry and in public transport. Therein, the surface to be treated is a surface, in particular, of furniture items, fittings, walls, floors or vehicles.

The method according to the invention can, however, also be used in areas where food, drinking water or other products for which a contamination with microorganisms is to be prevented are in contact with a surface. In these cases, the surface to be treated is, in particular, an inner surface of containers or pipes.

The present invention further relates to the use of collagen hydrolysate for treating a surface in order to improve the removability of microorganisms from the surface.

Advantages and preferred embodiments of the use according to the invention have already been described in relation to the method according to the invention.

The invention will now be described in greater detail based on the following examples and making reference to the drawings.

### Example 1

To demonstrate the effectiveness of the method according to the invention, the removability of Escherichia coli (E. coli) bacteria on a glass surface was investigated.

Eight slides made of glass having the dimensions 76×26 mm were rinsed with hot water and subsequently disinfected with isopropanol (slides No. 1 to 8). A treatment solution in accordance with the method according to the invention was applied to slides No. 5 to 8, specifically a solution of 5 wt % collagen hydrolysate in distilled water. Therein, 0.05 ml of the treatment solution was evenly distributed on each slide with a sterile spatula. The collagen hydrolysate is one such with a mean molecular weight of approximately 3000 Da, which is sold by the applicant under the designation NOVOTEC CB800.

For comparison, instead of the treatment solution, 0.05 ml of sterile tap water was applied in the same way to each of the slides No. 1 to 4.

On all eight slides, the treatment solution or water was subsequently allowed to dry in the air.

In order to produce a microbe suspension, a small quantity of a colony was removed with a sterile inoculation loop from a pure strain dish of E. coli and suspended in 5 ml of sterile mains water. From this microbe suspension, 3×0.5 μl was dropped onto each of the eight slides, the suspension having been homogenised afresh with a vortex mixer before the coating of a slide. Subsequently, the microbe suspension was air dried on the slides over a period of 17 hours.

After the drying, slides No. 2 and 6 were immersed for 30 seconds, and slides No. 4 and 8 for 60 seconds, in a glass beaker filled with distilled water, and then air dried again. Slides No. 1, 3, 5 and 7 remained untreated in this regard.

Finally, the bacteria were fixed on all the slides over a heating block at 70° C. for two minutes and thereafter stained with a methylene blue solution for one minute. The excess dye solution was poured away. The experiment was carried out in each case as a duplicate determination.

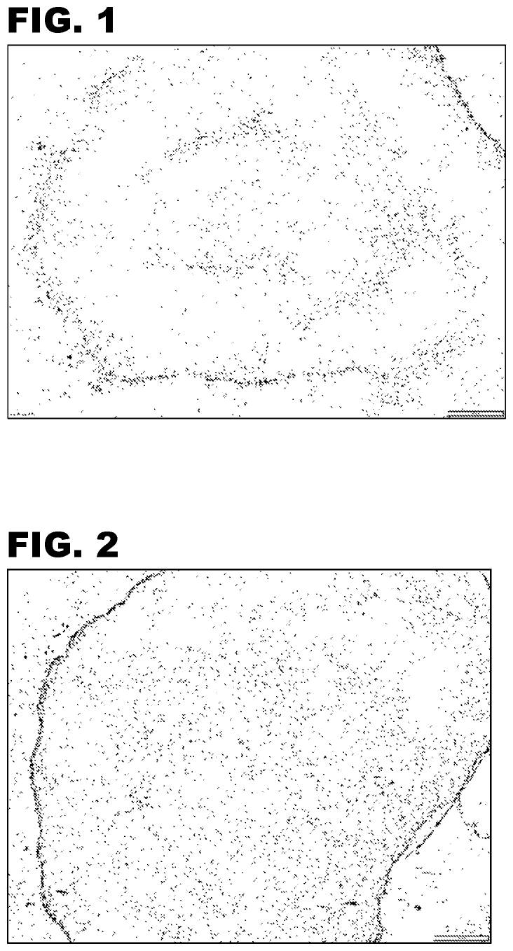

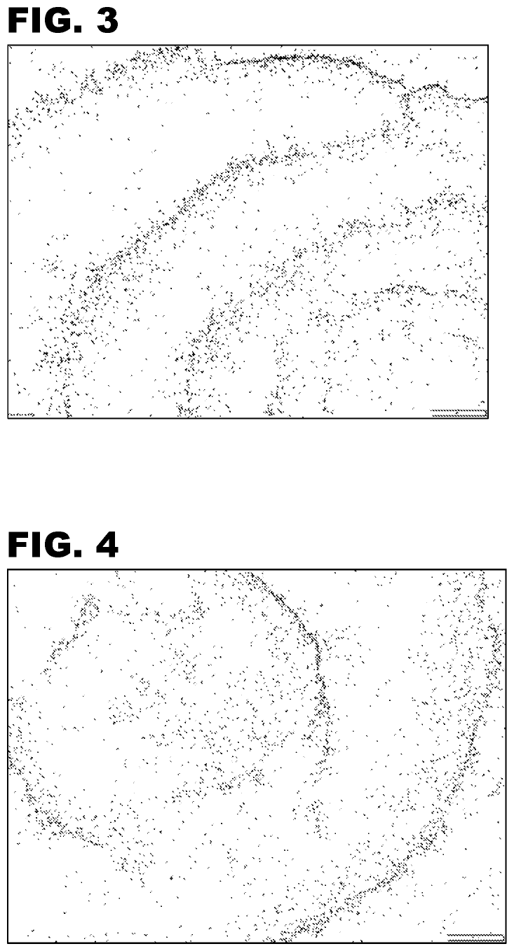

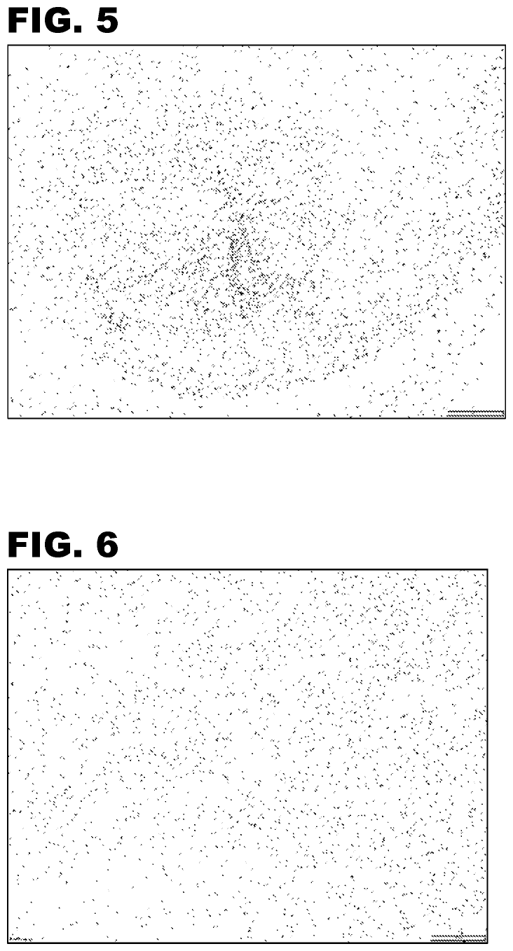

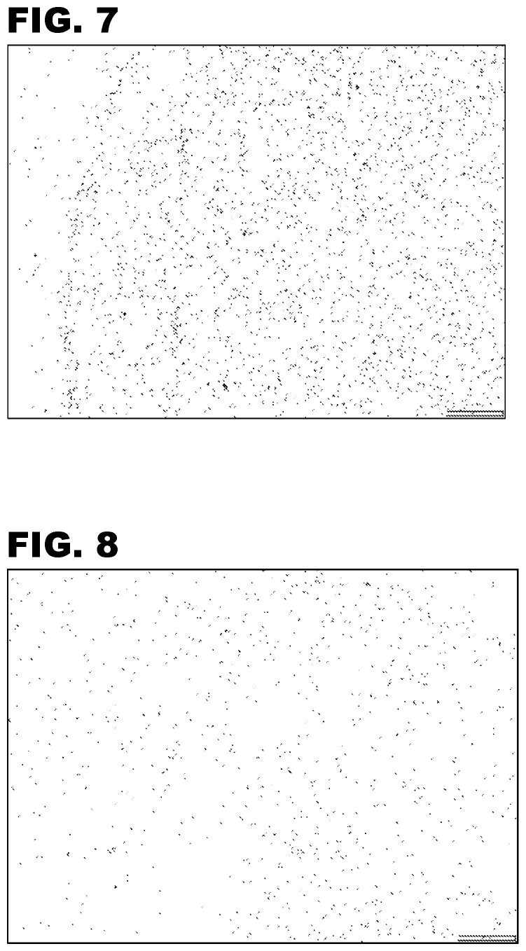

The slides were evaluated microscopically. FIGS. 1 to 8 each show microscopic images of the fixed and stained slides No. 1 to 8 at 100× magnification.

What is noticeable firstly is the different drying behaviour of the microbe suspension on the slides No. 5 to 8 treated according to the invention, as compared with the slides No. 1 to 4 previously treated only with water. Whereas the drop on the untreated slides dries from the outside inwardly, forming concentric rings of bacteria, the microbe suspension on the slides coated with collagen hydrolysate dries very homogeneously. This makes difficult a direct comparison of the microbe counts, for example, by automatic image analysis.

For quantitative evaluation of the slides, the microscopic images were therefore digitised and, by means of image analysis software, the number of the bacteria within an identical area in the interior of the drops was determined in each case a plurality of times. On the basis of the data thus obtained, the percentage reduction in the bacteria count due to the respective washing procedure (dipping in water) as compared with an unwashed slide was determined.

The results are reproduced in Table 1 below:

TABLE 1

| | | | Treated with | |
|---|---|---|---|---|
| | Untreated | | collagen hydrolysate | |
| | Slide | Bacteria count | Slide | Bacteria count |
| Not washed | No. 1 | Starting value | No. 5 | Starting value |
| Washed for 30 s | No. 2 | No reduction | No. 6 | Reduced by ca. 40% |
| Not washed | No. 3 | Starting value | No. 7 | Starting value |
| Washed for 60 s | No. 4 | No reduction | No. 8 | Reduced by ca. 60% |

A comparison of the values shows that the removal of bacteria from the glass surface by dipping the slide in water is significantly more effective if the surface has been treated with collagen hydrolysate before the application of the bacteria.

With this example, it is therefore confirmed that the removability of microorganisms from a surface can be improved by means of the method according to the invention.

1. A method for treating a surface in order to improve the removability of microorganisms from the surface, comprising:
applying a treatment solution onto the surface, wherein the treatment solution comprises an aqueous solution of a collagen hydrolysate; and
allowing the surface to dry.

2. The method according to claim 1, wherein the collagen hydrolysate is obtained by enzymatic hydrolysis of a collagen-containing starting material.

3. The method according to claim 1, wherein the collagen hydrolysate has a mean molecular weight from approximately 1,000 to approximately 7,000 Da.

4. The method according to claim 1, wherein the treatment solution comprises approximately 0.5 to approximately 8 wt % of the collagen hydrolysate.

5. The method according to claim 1, wherein the treatment solution is applied onto the surface in a quantity from approximately 10 to approximately 50 ml/m².

6. The method according to claim 1, wherein the treatment solution is spread or sprayed onto the surface.

7. The method according to claim 1, wherein the microorganisms comprise bacteria, fungi, algae and viruses.

8. The method according to claim 7, wherein the microorganisms are bacteria.

9. The method according to claim 1, wherein the treatment solution contains no biocides.

10. The method according to claim 9, wherein apart from the collagen hydrolysate, the treatment solution comprises no other active ingredients.

11. The method according to claim 1, wherein the treatment solution comprises further active agents which are selected from biocides, surfactants, phosphates, preserving agents, perfumes and/or excipients.

12. The method according to claim 11, wherein by means of the application of the treatment solution, microorganisms situated on the surface are removed, killed and/or deactivated.

13. The method according to claim 1, wherein the surface is a smooth surface.

14. The method according to claim 1, wherein the surface is a surface of furniture, fittings, walls, floors or vehicles.

15. The method according to claim 1, wherein the surface is an inner surface of containers or pipes.

16. (canceled)

17. The method according to claim 3, wherein the collagen hydrolysate has a mean molecular weight from approximately 2,000 to approximately 5,000 Da.

18. The method according to claim 4, wherein the treatment solution comprises approximately 2 to approximately 6 wt. % of the collagen hydrolysate.

19. The method according to claim 5, wherein the treatment solution is applied onto the surface in a quantity from approximately 20 to approximately 30 ml/m².

20. The method according to claim 10, wherein the treatment solution comprises no surfactants.

21. The method according to claim 13, wherein the surface is a surface of glass, plastics or metal.
